(19) United States
(12) Patent Application Publication
(10) Pub. No.: US 2008/0206156 A1
(43) Pub. Date: Aug. 28, 2008

(54) Continuous spray scalp therapy and dispensing systems for same

(76) Inventor: Peter J. Cronk, Moorestown, NJ (US)

Correspondence Address: DUANE MORRIS, LLP;IP DEPARTMENT, 30 SOUTH 17TH STREET, PHILADELPHIA, PA, 19103-4196, US

(21) Appl. No.: 11/677,777

(22) Filed: Feb. 22, 2007

(51) Int. Cl. A61K 9/12; A61K 31/506; A61M 11/00; A61P 17/14; A61K 8/04

(52) U.S. Cl. 424/45; 128/200.23; 424/59; 514/275

(57) ABSTRACT

The present invention relates to continuous spray medications involving the scalp, such as minoxidil, finesteride, copper peptides, DHT inhibitors and androgen receptor blockers, for example, in an amount sufficient to stimulate or maintain hair growth. The scalp medication is disposed within a pharmacologically acceptable carrier solution. In further embodiments, the spray scalp medication is dispensed within a product bag containing the medication in liquid form. The product bag is disposed within a container and includes a sheet of gas impervious material having a barrier layer therein. A gaseous propellant is also disposed within the container and is substantially separated from the medication. The valve assembly is selectively operated to produce a fine mist of said medication which is non-chilling, continuous and even.

### FIELD OF THE INVENTION

The present invention relates to scalp medications generally, and particularly those for treating alopecia.

### BACKGROUND OF THE INVENTION

Alopecia, the loss of hair in men and women, involves a gradual decrease of scalp hair density in adults, with a transformation of terminal to vellus hairs which become lost as a result of familial increased susceptibility of hair follicles to androgen secretion following puberty. Two areas of the scalp are commonly affected in men, as evidenced by a receding frontal and bilateral triangular temple hair lines, and a balding patch on the vertex, which may progress to complete male pattern baldness. In females, a diffuse partial hair loss in the central parietal area of the scalp with preservation of the frontal and temporal hair lines is most common.

One of the more common therapies for alopecia is minoxidil, which has been proven to be an effective hair growth stimulator that can both maintain and increase scalp hair counts. Minoxidil was first used as a patent antihypertensive drug. It was later discovered that Minoxidil had positive effects on hair growth when applied topically to the scalp. Minoxidil was first mass marketed as Rogain® lotion by Upjohn, Inc. for the treatment of hair loss in the late 1980s. Rogain® lotion still remains the only FDA approved topical medication for the treatment of hair loss in the United States, as of this date. The directions associated with traditional minoxidil lotions, such as Rogain® lotion, now manufactured by Pfizer Inc., is that the medication be applied twice a day for optimal results. These application times should be at least four hours apart, suggesting that the best times to apply the product would be right after the morning shower, and before bed at night. Unfortunately, the traditional 5% minoxidil Rogain® lotion utilizes as much as 50% by volume propylene glycol (20% v/v of propylene glycol for 2% minoxidil) as a vehicle for extending the delivery of the active medication to the scalp, and for ensuring that the applied minoxidil is evenly spread across the affected area and easily absorbed into the skin. Propylene glycol and ethanol alcohol are also used to solubilize minoxidil. The problem associated with smearing copious amounts of propylene glycol on one's scalp is that it leaves the hair and scalp with a greasy texture for an extended period of time after the application. The resulting appearance is often unflattering. It also leaves the hair looking matted down and even thinner than it really is, which may be considered an unfortunate step in the wrong direction for a hair loss treatment.

Another issue associated with propylene glycol is that it can irritate the user's scalp, resulting in itching, flaking, redness of the scalp, oiliness of the scalp, excessive dandruff, and even peeling in some cases. Because of these effects, the use of traditional minoxidil solution was not an option for many hair loss sufferers, and glycerine versions of the popular medication have been developed, available by prescription from Minoxidil.com.

More recently, Pfizer had introduced Rogain® Foam, which delivers the minoxidil into the scalp in a manner that is just as effective as the traditional propylene glycol based minoxidil lotions, but without some of the mess or unpleasantness. Rogain® Foam is designed to retain its “foamy” texture at room temperature, but quickly liquefies, spurring quick skin absorption at body temperature.

Unfortunately, fast skin absorption by Rogain® Foam means that the active ingredient of Rogain® Foam, minoxidil, will be readily absorbed into the hand of the user, where it clearly is not intended to be absorbed. Over-absorption of minoxidil can result in vision changes, chest pain and fast or irregular heart beat. Women exposed to over-absorption of minoxidil may experience additional unwanted facial hair growth. Additionally, minoxidil may pass from mother to child through breast milk. It is for these and other reasons that the Rogain® Foam package insert suggests that the user rinse his or her hand under cold water before applying the foam, to slow down absorption of the active ingredient into the hand. This, of course, requires another step (or two, if the user also dries his or her hand, as recommended by the manufacturer) in the application of the medication. It may also dilute the concentration if one does not immediately dry one's hand after wetting, and before applying the foam. In addition, hair does feel a little stiffer after the application of the Rogain® Foam and the smearing of the foamy application, with or without a wet hand, can also mat down hair styles, making them less natural looking.

Accordingly, there remains a need for further dispensing systems and methods for scalp medications, including minoxidil and other products, such as DHT inhibitors, finesteride (Propecia®), copper peptides (Tricomin® and Folligen®), other DHT inhibitors (Dutasteride®), all-around treatments or combinations (Proxiphen®) androgen receptor blockers (Spirondactone®), corticosteroids, vitamin A derivatives (Retin-A®), tretinoin, azelaic acid, zinc, B6, grape seed extract, nicotinate and/or progesterone, which are easy to use and provide a more natural appearance than conventionally applied propylene glycol-based and foam-based products delivery systems.

### SUMMARY OF THE INVENTION

In a first embodiment of the present invention, a continuous fine mist spray medication delivery system is provided. This system includes a selectively operable valve assembly affixed to a portable container containing a pressurized agent or pressure source and a product bag containing said spray medication. The product bag, also located in the container, includes a sheet of gas impervious material having a barrier layer therein. The gaseous propellant is substantially separated from the medication and its liquid carrier, at least prior to, and more preferably, prior to and after selective operation of the valve assembly to produce the fine mist. The continuous spray fine mist medication preferably comprises a hair growth stimulator or maintenance active ingredient, such as minoxidil, a DHT inhibitor, an androgen blocker, or other combinations thereof, in an amount sufficient to stimulate or maintain hair growth, or both, disposed within a pharmacologically acceptable solution, for example, alcohol, with or without propylene glycol, glycerin, dimethyl-isosorbide, or other “extenders” to permit better absorption.

It has been suggested that minoxidil is dose dependent, meaning that patients may have to increase the concentration of minoxidil over time in order to sustain the results. Consequently, it has been suggested that people should start using a lower strength formula, such as 2% v/v, and gradually move up the scale to 5% v/v, and then ultimately, to 12% v/v.

The present invention employs advanced spray technology to provide a quick, easy and effective application of scalp medications. In a preferred embodiment, a continuous spray of fine mist medication can be provided at every angle, even upside down, enabling application to hard to reach places like the vertex of the scalp. The preferred spray technology uses environmentally safe, bag-in-can technology to apply scalp medications, such as 2-15% v/v minoxidil (2,4-Diamino-6-piperidinopyrimidine 3-oxide) or DHT inhibitors, such as copper peptides or 5-alpha-reductase inhibitors, such as finesteride (such as Propecia® brand, available from Merck, which is not recommended for women who are or may potentially become pregnant), or dutasteride (such as Avodart®, available from GlaxoSmithKline), copper peptides (Tricomin® and Folligen® brands), all-around treatments or combinations (Proxiphen® brand), androgen receptor blockers (Spirondactone® brand), corticosteroids, Retin-A® brand, tretinoin, azelaic acid, zinc, B6, grape seed extract, nicotinate and/or progesterone. The delivery systems of this invention eliminate the uncomfortable twisting, reaching, and most of the hand-drug contact associated with applying conventional Rogain® liquid or Rogain® Foam to the back of one's scalp. Accordingly, the active scalp medications are easier to apply than Rogain® lotion, even when compared to Rogain® lotion dispensed from the pump-spray bottle, since the delivery system of this invention can preferably operate upside down without clogging or interruptions, and dispenses a preferred fine, even and continuous mist. Because this delivery system allows the scalp treatment to be applied evenly and continuously, directly from the container onto the scalp, with little or no need for rubbing, absorption into the hand of the user and unflattering matting of the hair due to rubbing can be eliminated.

The packaging of this invention also, preferably, maintains total product integrity and freshness, and extends the useful life of the medication, even lengthening shelf-life. In addition, by using a preferred bag-in-can system, the scalp medication can be separated from the pressurizing agent within a preferred hermetically sealed, multi-layered laminated pouch. The pouch can maintain total formulation integrity, and pure product, preferably, can be dispensed without contamination by propellants. The spray medication can have a quiet and non-chilling discharge, and allows all attitude (360°) dispensing. By using existing through-the-valve aerosol filling technology and equipment, the dispensing system of this invention can be designed to work with standard actuators and aluminum aerosol cans.

In a further embodiment of the present invention, a medication is provided which includes a continuous even mist, including liquid droplets, having an average diameter of about 1 nanometer to about 2000 micrometers, said mist containing minoxidil in an amount sufficient to stimulate or maintain hair growth, in which said minoxidil is disposed within a pharmacologically acceptable carrier solution.

In a preferred embodiment of this invention, the spray medication can be delivered quietly, under 50 decibels, and the continuous spray is, preferably, non-chilling upon contact with skin, or is approximately at ambient temperature. The valve assembly can include a two-way valve and the container may comprise an aluminum or steel can. The bag, desirably, includes at least one gusset so that it can expand when product is introduced and lay on the bottom of the container to relieve stress on the valve assembly.

The above and other features of the present invention will be better understood from the following detailed description of the preferred embodiments of the invention that is provided in connection with the accompanying drawing.

### BRIEF DESCRIPTION OF THE DRAWING

The accompanying drawing illustrates a preferred embodiment of the invention, as well as other information pertinent to the disclosure, in which:

FIG. 1 is a cross-sectional side elevational view showing a dispensing system (with a continuous spray in phantom) for housing the liquid medication of this invention.

### DETAILED DESCRIPTION

The present invention pertains to continuous spray medications, spray medication dispensing systems, and methods for treating alopecia, in which a continuous mist of a scalp medication, such as minoxidil, DHT inhibitors and/or androgen blockers, for example, is provided in an amount sufficient to stimulate or maintain hair growth, or both, for example. The minoxidil and/or other medication is disposed within a pharmacologically acceptable carrier solution.

In the preferred embodiments of this invention, the continuous mists are designed to generate liquid droplets that are readily absorbed through the dermis, especially scalp tissue. Mist particle sizes range from molecular clusters on the order of about 1 nanometer to about 2000 micrometers, and more preferably, the spray should provide good coverage of the surface area without running onto unwanted areas of the face and neck

The preferred delivery system of the present invention includes the ABS bag-in-can system, such as the system available from CCL Container. There preferred systems are hand held, portable, and completely self-contained. The ABS bag-in-can system separates the product from the pressurizing agent with a hermetically sealed, multi-layered laminated pouch “bag”. The bag maintains total formulation integrity, and only pure product, preferably, is dispensed, as opposed to other aerosol systems that can mix the product with the gaseous propellant. This maintains the total product integrity, such as alcohol mixtures, solutions and suspensions of minoxidil and fenesteride formulations, and potentially extends their shelf life. This system also has a quiet, non-chilling discharge, and allows for all-attitude (360°) dispensing, permitting “upside-down” dispensing, which is highly desirable for spraying one's scalp. While Rogain® lotion comes with its own pump spray dispenser, the dispenser can not work upside down and, consequently, is very awkward to use on the vertex of one's scalp. Pump-spray containers such as this one, are widely available and operate on the principle that when the nozzle pump is manually depressed, pressure is exerted on the liquid in the container, forcing it out of the spray nozzle. This pump dispenser is not continuous and produces a very uneven distribution of the product on one's scalp, generally depositing a shot of 0.5 ml or more of the ingredients, in a heavy squirt to a small area of the scalp, which tends to cause the liquid medication to run to where it is not needed, or worse, where it is definitely not recommended, such as on the face or the wearer's eyes. Moreover, conventional pump-spray dispensers such as these, attached to plastic bottles with plastic threads, are not hermetic, so the contents can be affected by environmental conditions, which, in some cases, reduce the efficacy of the medication, and/or is solvent (also referred to as the “extender” or “carrier” solution).

Alternatively, this invention can employ a piston barrier package (also available from CCL Container since 1991), which also assures separation of the scalp medication from the propellant or pressurizing agent, for maintaining the purity and integrity of the formulation throughout its consumer life span. The system provides smooth controlled and uniform discharge. In a piston barrier package, a piston is disposed in a preferred seamless tubular package between the propellant and the product. As the nozzle is activated, the piston is activated by the propellant pressure in the lower part of the container, to push out the product through the nozzle. The preferred CCL version of this system eliminates propellant bypass tendency common with other metal containers because of its seamless construction. It also comes with a specially engineered gassing hole at the bottom for improving sealing during gassing and plugging operations.

In certain formulations of the present invention, a clear, preferably non-rub, continuous spray of medication is provided. For example, an alcohol base with little or no propylene glycol can be used. Higher amounts of propylene glycol, such as the 50% by volume used in Rogain® lotion, can leave one's hair feeling flat and greasy. Glycerin and dimethyl-isosorbide, or combinations of glycerin, dimethyl-isosorbide, and/or propylene glycol (such as 50%/50% v/v or w/w glycerin-propylene glycol, 80%/20% v/v or w/w glycerin-propylene glycol, 80%/10%/10% v/v/v or w/w/w dimethyl-isosorbide, glycerin and propylene glycol, or the like) are more preferred for use as solvents and extenders for this invention. It has been reported that when dipropylene glycol additions to minoxidil solutions are less than 5% by weight, the retention effect of minoxidil in the dermis is insufficient, and when the amount is more than 40% by weight, the resulting preparation gives a bad feeling in use. See U.S. Pat. No. 6,255,313.

In the preferred embodiments of this invention, the minoxidil formulations can include other ingredients, such as butane, butylated-hydroxytoluene, cetyl alcohol, citric acid, fragrance, glycerin, isobutane, lactic acid, polysorbate 60, propane, purified water, SD alcohol 40B, stearyl alcohol. In other formulations, sunscreen additives can be added, such as butyl methoxydibenzoylmethane, homosalate, ethylhexyl-salicylate, benzophenone-3, diethylhexyl 2,6 naphthalate. If water resistance is required, acrylates/octylacrylamide copolymers (such as Dermacryl 79, available from National Starch) can be added.

In a further compositional embodiment, a minoxidil spray lotion formulation includes at least about 2% v/v or 0.1% w/w, and preferably, about 2-15% v/v or 0.1-10% w/w minoxidil in a carrier solution comprising butane, butylated hydroxytoluene, cetylalcohol, citrix acid, fragrance, glycerin, isobutane, lactic acid, polysorbate 60, propane, purified water, SD alcohol 40B and stearyl alcohol.

In still a further embodiment, a minoxidil spray lotion formulation includes about 2% v/v or 0.1% w/w, preferably about 2-15% v/v or 0.1-10% w/w minoxidil in a carrier solution comprising about 60% v/v or w/w alcohol, about 20-50% v/v or w/w propylene glycol or glycerin and purified water (balance). For greater sustained release, dipropylene glycol can be used, as shown in U.S. Pat. No. 6,255,313, which is hereby incorporated by reference. Gel formulations containing minoxidil are also contemplated by this invention and disclosed in U.S. Pat. No. 5,225,189, which is also hereby incorporated by reference.

The preparations of the present invention are preferably adjusted to a pH of 4-9, and more preferably about 5-8 when diluted with purified water.

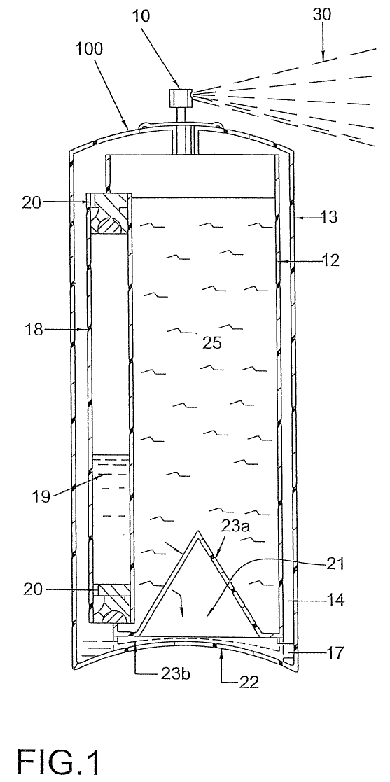

In more preferred embodiments of this invention, a continuous fine mist spray medication dispensing system 100 is provided, as shown in FIG. 1. The dispensing system includes a selectively operable valve assembly 10 affixed to a container containing a pressure source or pressurizing agent. A product bag 12 containing said medication 25 in a liquid form is also provided. The bag 12 is disposed within the container and includes a sheet of gas impervious material having a barrier layer therein. The gas impervious material helps to separate the medication 25 in liquid form from the pressure source or pressurizing agent located elsewhere in the container, so as to keep the medication fresh and efficacious.

The fine spray mist medication is preferably, delivered quietly, under 60 decibels (normal conversation, sewing machine, typewriter), and more preferably, less than 40 decibels, or most preferably, about 30 decibels or less (whisper, quiet library). It is a fine spray which is, preferably, non-chilling upon contact with skin, e.g., 98.6° F.±5° F. The valve assembly 10 is, preferably, a two-way valve. The container is preferably made of a metal, such as aluminum or steel. The bag 12 preferably includes at least one gusset 21 which expands from a position of 23a to a position 23b when the liquid medication 25 reaches a full level, at which time a fine mist 30 can be sprayed.

In a further embodiment of the present invention, a method of treating alopecia is provided comprising a delivery system which includes a valve assembly 10 affixed to a container 13 containing a pressure source or pressurized agent, and a product bag containing minoxidil in an amount sufficient to stimulate or maintain hair growth disposed within a pharmacologically acceptable carrier solution. The container 13 is then rotated, and the valve assembly 10 is located such that the valve assembly 10 is at least partially below a portion of the container 13, or even upside down. The method further includes continuously spraying a fine mist of said minoxidil and said carrier solution onto an alopecia affected area of the scalp. The applied solution is evenly distributed and no further action or rubbing is required, but some minor rubbing can be employed, if necessary, to reach spots missed by the initial application.

In the preferred embodiments of the present invention, the pharmacologically acceptable carrier solution contains less than about 50% v/v propylene glycol, and more preferably, less than about 20% v/v, and most preferably, less than about 5% v/v propylene glycol, or none at all. The carrier solution can also include, or alternatively comprise, glycerin, dimethyl-isosorbide, or a combination thereof, purified water and an alcohol, such as SD alcohol, ethanol or isopropanol.

In still a further embodiment of the present invention, a spray medication is provided which includes an even, continuous mist containing a scalp medication sufficient to stimulate or maintain hair growth. This embodiment of the scalp medication is disposed within a pharmacologically carrier solution. This spray medication is transparent on the scalp and does not need to be rubbed into the skin of the scalp to be effective in stimulating or maintaining hair growth. This medication embodiment also, preferably, contains less than 20% v/v propylene glycol for a 2% minoxidil solution.

With further reference to FIG. 1, the product dispensing system 100 incorporates a product dispensing bag 12 in accordance with the preferred embodiment of the present invention. The system includes a container 13 having external wall surface. A product dispensing bag 12 has a gusseted bottom 23 and sealed side portion or portions. The product dispensing bag 12 is also sealed around its top edge. A valve assembly 10 is attached to the product dispensing bag 12 via a valve connector extending into the top surface of the container 13. FIG. 1 illustrates a side elevational view of the product dispensing bag 12 showing the bag being partially filled. The gusset 23a can be opened, and then the expanded gusset 23b can rest on the dome-shaped bottom 22.

After the container 13 is sealed, product is injected into the product dispensing bag via the preferred two-way valve and valve connector. As the bag 12 fills, it expands, and the gusseted bottom 23 spreads along the surface 22 of the bottom of the container 13. The gusseted bottom 23 serves to prevent undue force on the seal between the valve 10 and the bag 12 when product is in the bag 12, because the mass of the product preferably rests on the bottom of the container, rather than being supported by the bag/valve interface if the bag hangs in free space. The gusseted bottom 23 controls the fill operation so that the bag fills more evenly and more fully. Furthermore, the gusset 23 improves bag fill capacity for a given container size. Preferably, the height of the gusset 23a (the distance between the bottom of the bag and the interior seam of the gusset) extends for approximately eighty percent (80%) of the radius of the container 13.

It is further necessary to provide a source of dispensing pressure in the container. In one method and system for providing a dispensing pressure that is regulated, a gas chamber consisting of the region in the closed container surrounding the product bag 12, can be charged to an initial dispensing pressure. The pre-charge exerts pressure on the product dispensing bag 12 so that when the valve 10 is activated, product is forced from the bag 12 and out of the container 13. Without regulation, the pressure may steadily decline as product is dispensed. Such a pre-charge can be provided by using compressed gas in the container surrounding the product bag, or by using a liquefied gas propellant.

The preferred product dispensing bag and dispensing system of the present invention is further described in U.S. Pat. Re. 35,540; 5,169,037 and 5,035,351, which are all hereby incorporated herein.

FIG. 1 further illustrates one type of a dispensing system configuration wherein the product dispensing bag 12 of the present invention is used with a pressure regulating mechanism. As described above, the product dispensing bag of the present invention can also be used in such systems as compressed gas or liquefied hydrocarbon systems or any product-in-bag dispensing system where a pressure source surrounding the bag is used to force product out of the bag, so that a continuous spray 30 can be generated.

A product bag 12 having a gusseted bottom is disposed within container walls 13. A gas generating chamber 14 is preferably defined by the area bounded by the container walls 13 and the exterior of the product bag 12. A first reactant 17, such as sodium bicarbonate, is disposed in a bottom of the container in the gas generating chamber 14 and a pressure regulating mechanism 18 is also disposed in the gas generating chamber. The pressure regulating mechanism 18 includes a second reactant 19 which can be a liquid reactant, such as citric acid or vinegar. In one embodiment, the pressure regulating mechanism is a hollow tube having check valves 20 disposed at either end. When the second reactant 19 combines with the first reactant, gas is generated within the gas generating chamber 14. The pressure regulating mechanism 18 is designed so that when a pressure outside of the tube exceeds a pressure inside of the tube, gas enters into the tube until pressure equilibrium is established. When the pressure inside of the tube exceeds the pressure outside of the tube, the second liquid reactant 19 is forced from the tube into the gas generating chamber 14 so as to react with the first reactant 17 to thereby generate gas within the gas generating chamber and reestablish pressure equilibrium between the pressure inside of the tube and the pressure surrounding the tube. The pressure generated in the gas generating chamber 14 places the product bag 12 under pressure and, hence, also places the product 25 disposed within the bag 12 under pressure as well. Thus, when valve 10 is activated so as to dispense product 25, product 25 is dispensed from the container under pressure produced in the gas generating chamber.

While, preferably, sodium bicarbonate is used as the first reactant, and citric acid as the second reactant, other reactants may be used. Also, solutions and slurries of the reactants may be used and the reactants may be interchanged, if desired.

The pressure regulating mechanism system 18 will be described in greater detail below. However, the tube is designed in such a manner so as to react with the first reactant 17 to maintain a substantially constant dispensing pressure throughout the dispensing of the entire product disposed in the product bag 12.

The initial pressure of the dispensing system is set when the product bag 12 is filled. As product 25 is introduced into the bag 12, the volume of the bag 12 expands, thereby reducing the volume of the gas generating chamber to, in turn, increase the pressure within that chamber. The increase in pressure of the chamber, in turn, results in an increase in the gas pressure within the pressure regulating mechanism 18. When the product bag 12 has been filled with product, a specific pressure is set in the gas generating chamber 14, and a gas pressure is also set in the pressure regulating mechanism 18 as equilibrium is established between the pressure inside and the pressure outside of that mechanism. The initial pressure is determined in accordance with the amount of product fill in conjunction with a given container size. Whenever the pressure in the gas generating chamber 14 drops due to the expulsion of product and the concomitant expansion of the volume of the gas generating chamber, the pressure regulating mechanism expels a predetermined amount of second liquid reactant 19 which mixes with the first reactant 17 and regenerates pressure to reestablish the initially charged pressure within the gas generation chamber. The amount of citric acid or vinegar, for example, discharged is determined by the pressure differential between the container and tube head space and the volume of gas in the tube. The act of filling the product bag 12 activates the pressure regulating system, charging it to a dispensing pressure. The pressure regulating system further controls the dispensing pressure over the course of dispensing the product from the container 13.

As further shown in FIG. 1, the product bag 12 has a gusseted end 21 and is a predetermined length, dependent upon the container size. More specifically, product dispensing bag 12 is of a length such that the presence of product in the bag 12 brings a base 23b (23a is the starting position) of the gusset 21 into contact with the bottom 22 of the container 13, which may be dome shaped. The gusset 21 serves to prevent undue force on a seal between the valve 10 and the bag 12 when product 25 is in the bag. Furthermore, the gusset 21 improves bag fill capacity of the product 25 for a given can size. Preferably, the height of the gusset 21 (distance between the bottom of the bag and interior seam of the gusset) extends for approximately 80% of the radius of the container.

It has also been determined that certain sizing relationships for the bag, the gusset, and the valve assembly further enhance the characteristics of the dispensing system. The bag height should be approximately equal to the difference between the inside can height (from the top rim of the container to the top surface of the bottom dome) and the valve height. Preferably, the material length is approximately equal to the sum of twice the bag height, and two times the gusset length.

Further reference is made to the following hypothetical examples of continuous spray medication compositions.

### EXAMPLES

A 2% v/v solution of minoxidil was prepared using the following carrier solutions:

| | |
|---|---|
| Ingredient | % v/v |

Carrier 1:

| | |
|---|---|
| SD alcohol, ethanol or isopropanol | 60.0 |
| propylene glycol, glycerin and/or dimethyl-isosorbide | 20.0 |
| purified water | 20.0 |

Carrier 2:

| | |
|---|---|
| SD alcohol, ethanol or isopropanol | 16.8 |
| propylene glycol, glycerin and/or dimethyl-isosorbide | 20.0 |
| purified water | 63.2 |

Procedure

In separate mixing vessels, add the carrier solutions. Then add milled minoxidil until they are in a 2% v/v (for higher concentrations of minoxidil, proportionately higher amounts of the propylene glycol, glycerin and/or dimethyl-isosorbide would be expected to be used) solution in each mixing vessel. Continue mixing until all ingredients are added and dissolved. Add concentrate to aerosol can and charge with propellant or propellant source.

Valving

| | | | |
|---|---|---|---|
| Vendor: | SeaquistPerfect | Vapor Tap: | None |
| Type: | XT 91 | Tubing ID: | 0.122″ |
| Stem Orifice: | 0.013″ | Actuator: | EUROSTAR ST |
| Spring: | 0.025″ SS | | VALVE |
| Body Orifice: | 0.13″ XT Standard | | 0.015″ DU3832 |

Although the invention has been described in terms of exemplary embodiments, it is not limited thereto. Rather, the appended claims should be construed broadly to include other variants and embodiments of the invention that may be made by those skilled in the art without departing from the scope and range of equivalents of the invention.

What is claimed is:

1. A fine mist spray delivery system in combination with a scalp medication, comprising:
a portable container having a gas impervious container wall;
a pressure source disposed within said container;
a selectively operable valve assembly affixed to said container; and
a product bag containing said scalp medication in liquid form, said product bag product disposed within said container and including a sheet of gas-impervious material having a barrier layer therein, said pressure source being substantially separated from said medication by at least said barrier layer, said scalp medication comprising a hair growth stimulator or hair growth maintenance active ingredient in an amount sufficient to stimulate or maintain hair growth disposed within a pharmacologically acceptable carrier solution, said scalp medication being dispensable at any angle, including when said value assembly is up-side down, to produce said fine mist of said scalp medication in a continuous fashion.

2. The delivery system of claim 1 wherein said active ingredient comprises 2-15% minoxidil.

3. The delivery system of claim 1 wherein said continuous spray is non-chilling (98.6° F.±5° F.) upon contact with skin, and is delivered quietly, under 60 decibels.

4. The delivery system of claim 1 wherein said valve assembly is a two-way valve.

5. The delivery system of claim 1 wherein said product bag comprises at least one gusset.

6. A method of treating alopecia, comprising:
providing a delivery system comprising a valve assembly affixed to a portable container containing a pressure source surrounding a product bag containing minoxidil in an amount sufficient to stimulate or maintain hair growth, said minoxidil disposed within a pharmacologically acceptable carrier solution;
rotating said container and said valve assembly such that said valve assembly is at least partially below a portion of said container;
actuating said valve assembly whereby said pressure source surrounding said product bag is used to force said minoxidil and said carrier solution out of the bag; and
continuously spraying a fine mist of said minoxidil and said carrier solution onto an alopecia affected area of a scalp.

7. The method of claim 6 wherein said pharmacologically acceptable carrier solution contains less than 20% propylene glycol.

8. The method of claim 7 wherein said pharmacologically acceptable carrier solution comprises 0% propylene glycol, but does contain alcohol, glycerin, or a combination thereof.

9. The method of claim 6 wherein said carrier solution further comprises a sunscreen.

10. A spray medication, comprising an even, continuous, non-chilling, mist containing a scalp medication sufficient to stimulate or maintain hair growth on a human scalp, said scalp medication disposed within a pharmacologically acceptable carrier solution, said scalp medication being non-chilling (98.6° F.±5° F.), transparent on said scalp, and not needing to be rubbed into the skin of said scalp to be effective in stimulating or maintaining said hair growth.

11. The spray medication of claim 10 wherein said carrier solution contains no propylene glycol.

12. The spray medication of claim 10 wherein said carrier solution comprises alcohol and glycerin.

13. The spray medication of claim 10 wherein said scalp medication comprises one or more of the group consisting of copper peptides, androgen receptor blockers, corticosteroids, vitamin A derivatives, tretinoin, azelaic acid, zinc, vitamin B6, grape seed extract, nicotinate, progesterone, or a combination thereof.

14. A spray medication comprising a continuous even mist including liquid droplets having an average diameter of about 1 nanometer to about 2000 micrometers, said mist containing minoxidil in an amount sufficient to stimulate or maintain hair growth, said minoxidil disposed within a pharmacologically acceptable carrier solution, said spray medication having a pH of about 4-9.

15. The spray medication of claim 14 wherein said mist comprises approximately 2-15% minoxidil in said carrier solution.

16. The spray medication of claim 15 wherein said carrier solution comprises an alcohol, an extender selected from the group consisting of propylene glycol, glycerin, dimethyl-isosorbide or a combination thereof, and purified water.

17. The spray medication of claim 14 wherein said mist further comprises a sunscreen.

18. The spray medication of claim 14 wherein said mist comprises a clear, no-rub formulation containing less than 20 wt % propylene glycol for a 2% minoxidil solution.

19. The spray medication of claim 14 further comprising a second medication selected from the group consisting of copper peptides, androgen receptor blockers, corticosteroids, vitamin A derivatives, tretinoin, azelaic acid, zinc, vitamin B6, grape seed extract, nicotinate, progesterone, or a combination thereof.

20. The spray medication of claim 14 wherein said continuous mist is generated by a pressurizing agent compressing on a product bag containing said medication.
